(19) United States
(12) Patent Application Publication
(10) Pub. No.: US 2008/0145364 A1
(43) Pub. Date: Jun. 19, 2008

(54) Method for High Throughput Screening for Anitbodies and Proteins Inducing Apoptosis

(75) Inventors: Sherman M. Weissman, New Haven, CT (US); Michael Snyder, New Haven, CT (US)

Correspondence Address: PATREA L. PABST;PABST PATENT GROUP LLP, 400 COLONY SQUARE, SUITE 1200, 1201 PEACHTREE STREET, ATLANTA, GA, 30361, US

(73) Assignee: Yale University

(21) Appl. No.: 11/816,074

(22) PCT Filed: Feb. 15, 2006
(86) PCT No.: PCT/US2006/005434

§ 371 Date: Aug. 10, 2007

Related U.S. Application Data
(60) Provisional application No. 60/652,878, filed on Feb. 15, 2005.
(60) Provisional application No. 60/726,296, filed on Oct. 13, 2005.

(51) Int. Cl. A61K 39/395; C40B 30/06; C07K 16/18; A61P 43/00; A61K 38/00; C07K 1/00

(52) U.S. Cl. 424/138.1; 506/10; 530/387.7; 530/300; 514/2

(57) ABSTRACT

High throughput assays used to identify antibodies and proteins that induce cell death are described herein. It is not necessary to identify the antigens the antibodies are reactive with prior to performing the assays. Instead, libraries of antibodies and proteins, including murine, human, humanized, single chain, and synthetic antibodies, are screened using high throughput assays to identify those antibodies and proteins which cause cell death. Standard technology is then used to screen for cell viability. Antibodies and proteins which induce apoptosis preferentially or exclusively of cancer cells are then isolated, characterized, and may be cloned. A method for cloning antibodies and proteins has been developed, which provides means for rapid identification of the antibody or protein and the gene encoding the antibody or protein, based on the presence of a “bar code” or “unique sequence.” A method for high throughput production of antibodies to human proteins has also been developed.

The present application claims priority under 35 U.S.C. § 119 to U.S. Ser. No. 60/652,878 filed Feb. 15, 2005, and U.S. Ser. No. 60/726,296 filed Oct. 13, 2005, both by Sherman M. Weissman and Michael Snyder.

### BACKGROUND OF THE INVENTION

The present invention is generally in the field of high throughput screening techniques to identify antibodies and other proteins that either associate tightly with specific targets or induce cell killing, including apoptosis, especially of cancer cells.

Antibodies have emerged as useful tools for research, diagnostics and therapeutics. In the last several years antibody-based therapies have revolutionized treatment approaches in oncology, transplant, and autoimmune diseases. The blockbuster drugs, RITUXAN®, HERCEPTIN®, and REMICADE® are just a few examples. Antibodies are routinely used in the diagnosis of many types of infectious agents, diseases and conditions. Moreover, the recent drive in proteomics to have affinity reagents generated against every human protein requires high throughput technologies for generating highly specific antibodies against a wide range of proteins.

Antibodies such as HERCEPTIN® induce apoptosis of the cells which they selectively bind to. Many such antibodies are known. In all cases, however, they require identification of a suitable antigen present on these cells, immunization of animals with the antigens, selection of those antibodies which bind to the antigens, then selection from this population of the antibodies which cause apoptosis. This is a long, labor intensive process that may yield only one or two candidates. These antibodies must then be modified; requiring further labor intensive studies to identify the genes encoding the antibodies, which then must be isolated and modified to yield a high affinity, humanized or single-chain antibody which is useful as a therapeutic.

High-throughput screening is a key link in the chain comprising the industrialized drug discovery paradigm. Today, many pharmaceutical companies are screening 100,000-300,000 or more compounds per screen to produce approximately 100-300 hits. On average, one or two of these become lead compound series. Larger screens of up to 1,000,000 compounds in several months may be required to generate something closer to five leads. Improvements in lead generation can also come from optimizing library diversity. Since the 1980s, improvements in screening technologies have resulted in throughputs that have increased from 10,000 assays per year to current levels, which can approach ultrahigh-throughput screening levels of more than 100,000 assays per day. High-throughput screening is evolving not only as a discrete activity, but as a perspective that is expanding backward toward target identification and validation and forward to converting assay hits to qualified leads via information generated either within screens or through downstream, high-throughput ADME (absorption, distribution, metabolism, and excretion) and toxicity testing.

High throughput screening has been used to identify and isolate antibodies, but only through binding of the antibodies to specific antigens, such as those present on a particular cell type, transformed or diseased cell, or a particular receptor or ligand. This has the disadvantage that one must have identified an antigen prior to generation and isolation of the antibodies.

It is therefore an object of the present invention to provide a high throughput method for identification of antibodies inducing apoptosis or cell death, without first identifying the antigen to which the antibody is reactive, and antibodies obtained using the method.

It is another object of the present invention to provide high throughput methods for identification of other proteins that induce apoptosis or cell death, without first identifying the proteins, and the resulting identified proteins.

It is a further object of the present invention to provide methods for rapidly cloning genes encoding antibodies and other proteins.

It is a further object of the present invention to provide methods for high-throughput production of antibodies.

### SUMMARY OF THE INVENTION

High throughput assays are used to identify antibodies and proteins that induce apoptosis. It is not necessary to identify the antigens the antibodies are reactive with prior to performing the assays. Instead, libraries of antibodies and proteins, including murine, human, humanized, single chain, and synthetic antibodies, are screened using high throughput assays to identify those antibodies and proteins which cause cell death. The assays are typically performed using a microarray that contains wells of one or more cell types, including both normal controls and cells to be killed, usually cancer cells. The microarray will typically include a number of different normal and cancer cell types since antibodies and proteins may induce apoptosis only in certain types of cancer cells, or at certain stages of the cancer. Standard technology is then used to screen for cell viability. Antibodies and proteins which induce apoptosis, preferentially or exclusively of cancer cells, are then isolated, characterized, and may be cloned. Alternatively, a mixture of antibodies is applied to a cell type, the apoptotic or dead cells are isolated, and the antibodies associated with these cells are then isolated, cloned, and confirmed for their ability to induce cell death.

A method for cloning of antibodies and proteins has been developed. The method involves insertion of a “bar code” or “unique tag” into the gene encoding the antibody or protein, where the tag encodes a unique amino acid sequence in the antibody or protein. This provides a means for rapid identification of the antibody or protein and the gene encoding the antibody or protein, based on the presence of the unique code.

A method for high throughput production of antibodies to human proteins has also been developed. Briefly the method contains the steps of interacting antibody libraries with thousands of different proteins produced using high throughput techniques and displayed in a multi-well format. The antigens are exposed to antibody libraries for an extended period of time to allow each antibody to bind to the antigen to which it has the highest affinity. Bound antibodies are then identified, characterized, and may be cloned. In a preferred embodiment, each member in the antibody and/or protein library contains a “bar code” or “unique tag” as described herein for rapid cloning and identification of the antibody and/or rapid identification of the protein that is bound to an antibody.

### DETAILED DESCRIPTION OF THE INVENTION

### Definitions

Normal cells as used herein are cells present in the body, an organ or a tissue, or isolated in cell culture that are not diseased or transformed.

Abnormal cells as used herein are cells that are diseased or transformed. These may be in an individual, organ, tissue or isolated in cell culture.

As used herein, high throughput assays are processes in which batches of compounds are tested for binding activity or biological activity against target molecules. Test compounds can act as inhibitors of target enzymes, as competitors for binding of a natural ligand to its receptor, or as agonists or antagonists for receptor-mediated intracellular processes. High-throughput assays are used to screen large numbers of compounds rapidly and in parallel.

Apoptosis is a genetically determined process of intracellular cell destruction postulated to exist and to be activated by a stimulus or by the removal of a suppressing agent or stimulus in order to explain the orderly breakdown and elimination of superfluous or unwanted cells. Programmed cell death is the death of cells by a specific sequence of events triggered in the course of normal development or as a means of preserving normal function. Apoptosis signifies a process in which certain signals lead cells to self-destruct.

As used herein, the term “antibody” includes immunoglobulin molecules and immunologically active portions of immunoglobulin molecules, i.e., molecules that contain an antigen binding site which specifically binds an antigen. Structurally, the simplest naturally occurring antibody (IgG) comprises four polypeptide chains, two heavy (H) chains and two light (L) chains inter-connected by disulfide bonds. The light chains exist in two distinct forms called kappa (κ) and lambda (λ). Each chain has a constant region (C) and a variable region (V). Each chain is organized into a series of domains. The light chains have two domains, corresponding to the C region and the other to the V region. The heavy chains have four domains, one corresponding to the V region and three domains (1, 2 and 3) in the C region. The naturally occurring antibody has two arms (each arm being a Fab region), each of which comprises a V_{L} and a V_{H} region associated with each other. It is this pair of V regions (V_{L} and V_{H}) that differ from one antibody to another (owing to amino acid sequence variations). The variable domains for each of the heavy and light chains have the same general structure, including four framework regions (FRs), whose sequences are relatively conserved, connected by three hypervariable or complementarity determining regions (CDRs). The variable region of each chain can typically be represented by the general formula FR1-CDR1FR2CDR2-FR3-CDR3-FR4. The CDRs for a particular variable region are held in close proximity to one and other by the framework regions, and with the CDRs from the other chain and which together are responsible for recognizing the antigen and providing an antigen-binding site (ABS).

Examples of binding fragments encompassed within the term antibody include (i) the Fab fragment consisting of the V_{L}, V_{H}, C_{L} and CH₁, domains; (ii) the Fd fragment consisting of the V_{H} and C_{H1} domains; (iii) the Fv fragment consisting of the V_{L} and V_{H} domains of a single arm of an antibody, (iv) the dAb fragment (Ward et al., (1989) Nature 341:544-546) which consists of a V_{H} domain; (v) isolated CDR regions; and (vi) F(ab′)₂ fragments, a bivalent fragment comprising two Fab fragments linked by a disulfide bridge at the hinge region. Although the two domains of the Fv fragment are coded for by separate genes, it has proved possible to make a synthetic linker that enables them to be made as a single protein chain (known as single chain Fv (scFv); Bird et al. (1988) Science 242:423-426; and Huston et al. (1988) PNAS 85:5879-5883) by recombinant methods. Such single chain antibodies are also encompassed within the term “antibody”.

As used herein, the term “antibody or protein library” refers to a plurality of antibodies and proteins comprising a plurality of unique immunoglobulins or antibody chains (e.g., heavy or light chains). In preferred embodiments, antibody or protein libraries comprise at least 10², more preferably, at least 10³, even more preferably at least 10⁴, and still more preferably, at least 10⁵ unique antibodies or antibody chains or proteins.

### I. High Throughput Production of Antibodies and Proteins

Antibodies and protein combinations for hundreds of proteins can be tested in parallel using protein arrays and antibody or protein libraries. Briefly, thousands of different proteins are produced using high throughput techniques and displayed in a multiwell format (e.g 96 to 1536 wells). The antigens thus displayed are exposed to antibody libraries for extended periods of time, typically two to twenty-four hours, as necessary for binding at one or more affinities. This allows each antibody in the library to bind the antigen to which it has highest affinity. Bound antibodies and proteins are identified using one of a variety of approaches. For example, when using a phage display method antibodies or proteins are expressed in phage as fusions with a phage surface protein, resulting in the antibodies or proteins being displayed on the surface of the phage. A library of phage expressing different binding moieties is produced and bound to immobilized, target proteins in high throughput fashion. Phage with high affinity for target proteins are then isolated. Serial passages may be necessary to enrich for antibodies and proteins of interest. To do this the selected phage from one round are re-grown in bacteria, the new enriched phage culture is harvested, bound again to immobilized target proteins and the newly selected phage are re-isolated. The isolated phage can be amplified for further testing and the sequence of the binding region determined. Other methods known in the art for displaying antibodies or proteins can also be used in addition to phage display.

Several types of antibody or protein libraries can be used for screening, including single chain, phage display, and potentially a two chain antibody library generated through a strategy described below. Ideally humanized antibodies and proteins will be used so that they can be used for therapeutic purposes. Antibody and protein libraries are commercially available from a number of sources. For example, Cambridge Antibody, Cambridge, United Kingdom, has a library of antibodies obtained from the blood of healthy people. Genetastix Corporation also has a human antibody library. A single chain antibody library is available from Pacific northwest Laboratory, Richland, Wash. A synthetic human combinatorial library is available from MorphoSys AG, Munich Germany.

In initial libraries the concentration of any specific antibody or protein inducing apoptosis will be very low. An improvement of signal to background can be obtained by selecting with a group of proteins immobilized together at high density on a single substrate (membrane spot, bead, etc.) Individual antibodies and proteins will be deconvoluted to individual substrates at subsequent steps. This may be particularly effective for generating antibodies against closely related members of protein families. For example, to prepare antibodies specific for a member of the C/EBP transcription factor family, such as C/EBP beta, single phage isolated from a mixture of the C/EBP factors would be tested separately against each factor and antibodies that did not react with other members of the family would be selected.

Widely used principles to select for highest affinity antibodies are to incubate for a long time so as to diminish kinetic trapping and approach equilibrium representing relative affinities by relative amounts of bound antibody, or else to wash bound material through several half lives to enrich for products with lowest off time. Problems of avidity as contrasted with affinity are commonly dealt with by using single chain antibodies. A difficulty with these methods is that high affinity antibodies have slow off times. One may select for higher affinity antibodies by loosening binding, for example, by adding glycols and incubating at very low temperatures to reduce hydrophobic interactions, by washing in higher salt for hydrophilic interactions, and by heating to temperatures approaching destabilization temperatures for antibodies, to energetically favor denatured states that may not bind.

The binding reaction can be detected in emulsion droplets by using antibodies fused to half of intein or half of fluorescent protein (GFP) and targets fused to the other half of the respective product. In the case of intein reaction, one can use binding and intein recombination to generate signaling molecules such as alkaline phosphatase, FACS emulsion droplets containing microbeads can be used to separate and capture droplets expressing both the chromophore and antibody then be arrayed. The protein tags on the antibodies in individual beads can then be analyzed by mass spectroscopy, mRNA in the positive droplets can be amplified by PCR or phage from the droplets arrayed.

Once antibodies of interest are identified, their specificity is assessed using high content protein arrays containing many types of proteins. Protein arrays consisting of several thousands of distinct proteins spotted in addressable format on modified glass surfaces can be fabricated or purchased from commercial vendors such as Invitrogen, Carlsbad Calif. Antibodies of interest are incubated with the protein array to allow antibody binding to protein targets. The slides are washed and specific antibody-protein interactions detected using a fluorescently labeled secondary antibody directed against the primary antibody, or by directly coupling a fluorophore to the primary antibody. Slides are then scanned and fluorescent protein spots identified, revealing the identity of the interacting proteins. The antibodies can also be tested against a panel of tumors to determine if any are of potential use as diagnostic markers. For example, tissue microarrays consisting of up to 600 addressable samples of biopsies from a range of cancer types and tissues can be screened for specific antibody binding and compared to similar screens of healthy tissue biopsies. Such tissue microarrays are available, for example, from Yale Cancer Center Tissue Microarray Facility, New Haven, Conn. Higher specificity can be achieved by linking low affinity antibodies that recognize different epitopes together. For example, if antibodies are available for two separate epitopes on the same protein, then a chimeric antibody can be made expressing variable regions from the heavy and light chains of each antibody. In principle such a hybrid antibody would have an affinity for the protein equal to the product of the affinities of the two original antibodies.

### II. Construction of Libraries of Antibody Genes or Other Polypeptides, Modifications of Current Approaches

Protein “bar codes” can be constructed by including in the construct coding sequences for protein tags, so that the resulting protein can be cleaved with specific protease(s) and generate a signature peptide whose sequence or composition will predict PCR primers that will selectively amplify the coding region for that protein out of a complex mixture. In this manner, a unique tag to every protein in a mixture can be generated. Triplet nucleotide codons each encoding a different amino acid are used. For example, a group of triplet oligonucleotides may be ligated together in random fashion and cloned as single molecules into a vector that will be used for antibody expression. A unique codon or series of unique codons are thus inserted into the DNA sequence or construct of each library member. Multiple tags are randomly inserted into a mixture of the DNA sequences of the library members. This results in each library member having a unique tag. Each codon is unique, but has a known amino acid and DNA sequence. The polypeptide containing the amino acid encoded by the unique codon is cleaved, and released from the antibody or protein. The clone can be identified depending on the size of the polypeptides. The content of the cleaved or uncleaved polypeptide can be determined by mass spectrometry (MS) since each amino acid sequence has a precise weight. For polypeptides with similar masses, isotopes can be used to manipulate the weight. Examples of amino acid isotopes include, but are not limited to, amino acids fully labeled with ¹³C and ¹⁵N and amino acids labeled selectively with ¹³C, ²H, and/or ¹⁵N.

A particular advantage of the use of mass spectrometry for polypeptide sequence determination is the speed, reproducibility, low cost and automation associated with mass spectrometry, especially in comparison to gel electrophoresis. Mass spectrometry has the ability to detect variances between two or more related polynucleotides to differentiate masses within a few or even less than one atomic mass unit (amu) of each other. Permitting such detection without the need for determining the complete sequence of the polypeptides being compared; i.e., the masses of the oligonucleotides provides the polypeptide or polynucleotide content. PCR primers are designed according to the identified sequence of the polynucleotide or polypeptide in order to selectively amplify the coding region for each uniquely tagged protein in a complex mixture.

For synthesis of the bar code region or unique tag, nucleotide triplets are assembled such that a single triplet or triplex codon is used for each amino acid, and therefore an amino acid at a certain position indicates that there is a particular nucleotide triplet at the corresponding region of mRNA. This procedure may work best with the sensitivity and precision of Fourier Transform Ion Cyclotron Resonance (FTICR) mass spectrometry, which has the ability to measure the mass of polypeptides with very high accuracy. By determining the mass of the polypeptide the sequence of the polypeptide can be determined.

In the simplest case, triplets of nucleotides will be added to each other successively by standard procedures for randomizing. The nucleotide tag can be generated by adding a mixture of triplets, or by synthesizing on beads. In this example, a particular triplet is added to a well containing beads, which are re-distributed among a number of wells after each addition step, followed by addition of a different triplet to each well. In this case mass spectrometry (“MS”) sequencing is used for each peptide after the protein selection.

In another embodiment, a “random” coding sequence contains two unique tags, such that two segments of “random” coding sequence are synthesized, either to be separated as peptides by use of two proteases or because of linking fixed amino acids, to run in two size ranges in MS. The sequences of these two segments of the “random” coding sequence are used to design PCR primers to generate the coding region. Use of two successive primers can both give a larger space for bar code coverage and increased specificity at the PCR recovery stage. As one example, two primers predicted from the segments of the bar code or unique codon can be used for inverse PCR on a plasmid mixture to recover coding sequences of desired protein.

Protein bar coding may offer an alternative to protein array screening for some purposes. For example, if one prepared a mixture of many tagged proteins in vitro, then selected in solution for binding to a particular immobilized protein, FTICR analysis of trapped bar codes will give information as to which proteins were bound.

### In Vitro Synthesis of Antibodies and Proteins

Lipid emulsions (LEs) are heterogenous dispersions of two immiscible liquids (oil-in-water or water-in-oil). Water-in-oil (w/o) emulsions can be used to compartmentalize and select large gene libraries. The aqueous droplets of the w/o emulsion function as cell-like compartments in each of which one or more genes are transcribed and translated to give multiple copies of the protein (e.g., an enzyme) it encodes. Compartmentalization ensures that the gene, the protein it encodes, and the products of the activity of this protein remain linked. Water-in-oil (W/O) emulsions are also used for PCR. These droplets function as micro-reactors and allow the effective concentration of template DNA to be increased, even for low concentrations of template DNA.

Synthesis of antibodies and proteins in emulsion droplets each containing one to a few templates can be used, for example, in combination with templates that separately synthesize light and heavy chains, to assure efficient yield of desired combinations of chains. In one embodiment, water-in-oil emulsion droplets contain one or more templates encoding a single light chain of an antibody and one or more templates of a single heavy chain of an antibody in order to generate a two chain antibody. Alternatively, one or more templates containing the sequence encoding the light chain and the heavy chain in emulsion droplets are used to generate a two chain antibody. In this embodiment, simultaneous synthesis of the light and heavy chains of the antibody promotes association of the light and heavy chains to produce functional antibodies.

In another embodiment, the complement of a bar code or protein tag can be attached to beads in multiple copies by the method of selective redistribution of subsets of beads after each synthesis step. After this each bead, in an emulsion droplet, will capture multiple copies of the same template from a cloned library or transcripts of a cloned library. Since PCR can be performed in emulsion droplets, additional copies of this antibody or protein can be generated by PCR. Each bead will then contain multiple copies of the same antibody or protein whether by mRNA capture or by synthesis in emulsion droplets, each containing a single bead.

After recovery of bar coded proteins, optionally linked to their templates, limited reannealing of bar code primer amplified material or recovered cDNA from enriched mRNA templates will selectively enrich most abundant species of bar coded or tagged template.

Single chain antibodies can be converted to two chain antibodies by constructing light and heavy chain coding regions flanked by att or lox sites so that they can be rapidly and in bulk transferred to vector expressing the two chains separately so as to generate standard two chain antibody. Alternatively a single att site can be used and a DNA fragment inserted with translation terminator, new promoter or Internal Ribosome Entry Site (IRES) and new translation imitator, etc.

### III. Identification of Therapeutic Antibodies and Proteins

High throughput methods are used to identify antibodies and proteins that induce apoptosis of one or more cell populations, typically cancer cells. In addition to high throughput identification of antibodies and proteins utilizing protein arrays, antibody or protein libraries can be screened to identify those that induce apoptosis of specific cancer cell types. Briefly, antibody and protein libraries are incubated with one or more different types of cancer cells. Those wells where the cells under apoptosis are isolated, and the antibodies and proteins further tested against control cells, especially normal cells, and other types of tumor cells. Alternatively, apoptotic or dying cells can be separated from a mixture of cells incubated with an antibody library by FACS. The antibodies bound to the apoptotic or dying cells are then isolated, cloned, and confirmed for their ability to induce cell death. The apoptotic or dying cells can be selectively marked by staining with antibodies against annexin V (a surface molecule on apoptotic cells) or by staining with propidium iodide to identify cells with sub-diploid DNA content (another characteristic of apoptotic cells). The desired cells expressing either marker could be selected by preparative FACS. As one alternative method, annexin V positive cells could be recovered in bulk by use of magnetic beads coated with an antibody against annexin V.

Identification of antibodies that induce apoptosis in cancer cells selected by their ability to bind a specific, known antigen has been described (see, e.g. U.S. Pat. Nos. 6,416,958; 6,252,050; and 6,458,356).

Identification of antibodies and proteins that induce apoptosis by exposing a multitude of antibodies and proteins directly to cancer cells using a high-throughput method is described herein. In one embodiment cell surface proteins are assembled onto a chip or a bead using baculovirus. After initial enrichment for antibodies and proteins that react with cell surface molecules the resulting library can be used for subsequent screening procedures. Initially, antibodies and proteins are screened for induction of apoptosis in normal cells and such antibodies and proteins are depleted. This is followed by a screen for the ability of the remaining antibodies and proteins to induce apoptosis in specific cancer cell types. It also may be necessary to sensitize the cells to apoptosis as described below.

In one embodiment, a sensitizer such as a cytotoxic compound or immunomodulatory compound is added to the microarray during screening. Examples of cytotoxic compounds include known chemotherapeutic agents such as BCN, cisplatin, taxol and antibodies such as HER2. Other compounds include reagents such as hydrogen peroxide and desferoxamine which cause hypoxia. Examples of useful immunomodulatory compounds include cytokines such as tumor necrosis factor, interferon gamma, and interleukin-2, and inhibitors of NFKβ. The latter can also include compounds that initiate cell death by activating the complement cascade such as by immune complex deposition on the cells that are to be killed. This provides a means for screening for antibodies or proteins inducing apoptosis in combination with the cytotoxic compound or immunostimulatory compound, where the antibodies or proteins might not induce apoptosis when administered alone.

A number of techniques well known to one of skill in the art can be used to identify cells that undergo apoptosis. In the simplest case, cells undergoing apoptosis are identified by staining with annexin antibodies and FACS analysis. A number of assays are commercially available to screen cells for apoptosis following exposure to the antibodies and proteins. To achieve a high enough concentration of antibodies and proteins it may be necessary to have many pools of low complexity libraries (e.g. 100-1000 antibodies and proteins per pool).

For example, Beckman Coulter has a suitable system, the high-throughput cell-based apoptosis assay using CellProbe HT Caspase 3/7 whole cell assay on Beckman Coulter Biomek 2000 Laboratory Automation Workstation.

BioVision Incorporated, Mountain View, Calif., sells a kit that utilizes bioluminescent detection of the ATP level via luciferase catalyzed reaction for a rapid screening of apoptosis and cell viability in mammalian cells. The assay can be done directly in culture plates requiring no harvest/washing/or sample preparations and can be fully automatic for high throughput (10 seconds/sample) and is highly sensitive (detects 10-100 mammalian cells/well). designed to detect ADP/ATP ratios for a rapid screening of apoptosis/necrosis/growth arrest/cell proliferation simultaneously in mammalian cells. Offers highly consistent results and with excellent correlation to other apoptosis markers (e.g. TUNEL-based assays and caspase assays). Assay can be fully automatic for high throughput (10 seconds/sample) and is highly sensitive (detects 10-100 cells/well).

Molecular Probes Inc, Eugene, Oreg. sells a kit that measures membrane permeability due to necrosis or apoptosis, the Vybrant Apoptosis Assay Kit #4.

In one embodiment, a cell surface protein assembly is constructed on a chip or bead, using baculovirus. After initial enrichment for antibodies and proteins reacting with cell surface molecules the resulting library can be used for less high complexity procedures. For example, screening can be done with individual antibody templates or template collections on beads in droplets that also contain cells. After synthesis/incubation droplets can be converted to single aqueous phase and apoptotic cells recovered by preparative FACS, and the antibodies and proteins enriched for these cells decoded as above. Alternatively apoptotic cells will be captured on bead and screened by caspase substrate or other colorimetric indicator of apoptosis.

In an alternative approach, various dilutions of the antibody or protein preparations are placed with cells in a single liquid culture, apoptotic cells isolated and antibodies and proteins binding to these cells recovered, on the assumption that there will not be enough effective apoptosis producing antibodies and proteins of any type to affect all the cells. In either case, antibodies and proteins against apoptotic cells of an irrelevant lineage will be deleted.

In one embodiment screening for antibodies and proteins containing protein bar codes is carried out using individual templates or template collections as described on beads in emulsion droplets that also contain cells. Following antibody or protein synthesis the droplets are converted to a single aqueous phase and apoptotic cells are recovered via FACS. The antibodies and proteins enriched for these cells are decoded as described above. Alternatively, following antibody or protein synthesis the apoptotic cells are captured on beads and screened for caspase substrate activation or another calorimetric indicator of apoptosis and the antibodies and proteins are decoded as described above.

In another embodiment various dilutions of the antibody or protein preparations are administered to cells in a single liquid culture. Apoptotic cells are isolated and antibodies binding to these cells are recovered.

In these embodiments antibodies and proteins that induce apoptosis in normal cells are depleted prior to identification of antibodies and proteins that induce apoptosis specifically in cancer cells.

In another embodiment antibody and protein libraries can also be incubated with one or more different types of cancer cells to identify antibodies that induce cell death via the complement cascade or cell death by Natural Killer (NK) cells. These antibodies and proteins are also further tested against control cells, especially normal cells, and other types of tumor cells.

The complement cascade involves a group of proteins which work with (complement) antibody activity to eliminate pathogens. The classical complement cascade is activated by antigen-antibody complexes. When the variable region of IgM or IgG binds antigen, the conformation of the Fc (constant) region is altered, allowing the C1q protein to bind, which triggers activation of the cascade. The endpoint is formation of a membrane attack complex (MAC), which inserts into lipid membranes of bacteria or eukaryotic cells and causes osmotic lysis. Complement binds to specific receptors on various cell types to mediate its inflammatory activities. The best characterized of these receptors is CR1, which binds the opsonin fragments C3b and C4b and promotes phagocytosis and clearance of antigen-antibody complexes in combination with antibody binding to FcR. A receptor for C1q also promotes immune complex binding to phagocytes.

In this embodiment, antibodies are incubated with cancer cells along with the factors involved in the classical complement pathway to identify antibodies induce cell death via the complement pathway. Preferably, the antibody library is usually first screened on control cells to eliminate antibodies which induce cell death in normal cells. Once antibodies that induce cell death are identified in wells containing the cancer cells or are isolated by FACS analysis. These antibodies are then isolated, cloned, and confirmed for their ability to induce cell death via the complement cascade.

Natural Killer (NK) cells are lethal lymphocytes. Like cytotoxic T cells, they contain granules filled with potent chemicals. NK cells are designed to kill certain mutant cells and virus-infected cells in one of two ways: (1) they kill cells to which antibody molecules have attached through a process called antibody-dependent cellular cytotoxicity (ADCC) or (2) they kill cells lacking MHC-I molecules on their surface. In ADCC, the Fab portion of the antibody binds to epitopes on the “foreign” cell. The NK cell then binds to the Fc portion of the antibody. The NK cell is then able to contact the cell and release pore-forming proteins called performs, proteolytic enzymes called granzymes, and chemokines. Granzymes pass through the pores and activate the enzymes that lead to apoptosis of the infected cell by means of destruction of its structural cytoskeleton proteins and by chromosomal degradation. As a result, the cell breaks into fragments that are subsequently removed by phagocytes. Perforins can also sometimes result in cell lysis. NK cells also cause death by inducing apoptosis in the target. The cytokine TNF alpha is released by the NK cells and may be involved in this process.

In this embodiment, the antibodies are incubated with cancer cells along with NK cells to identify antibodies that induce cell death via NK cells. Preferably, the antibody library is usually first screened on control cells to eliminate antibodies which induce cell death in normal cells. Once antibodies that induce cell death are identified in wells containing the cancer cells or are isolated by FACS analysis. These antibodies are then isolated, cloned, and confirmed for their ability to induce cell death by NK cells.

Once the antibodies and proteins of interest have been identified, the gene(s) encoding the antibody or protein are isolated using primers designed based on the antibody variable region or the protein tag inserted as discussed above.

### IV. Therapeutic Applications

Antibodies and proteins that are identified using the methodology above are further tested for specificity and affinity, to insure that the antibodies and proteins only induce apoptosis in the desired cell population. These may then be modified to humanize, increase affinity and/or prepare recombinantly.

Once the antibodies and proteins have been confirmed to have the desired specificity and affinity, they may be formulated for use as a pharmaceutical. Typically antibodies and proteins are suspended in a buffered saline solution and injected intravenously for use. These will be administered in a dosage and for a period of time effective to induce tumor death. The appropriate dosages are determined using standard techniques based on effective concentrations in cell culture and animal studies.

Antibodies and proteins identified by the methods described above may be administered in combination to improve efficacy of treatment. For example, two antibodies and proteins of low affinity and reactive with different epitopes may be administered to a patient to increase induction of apoptosis in cancer cells.

The mechanism of apoptosis is remarkably conserved throughout evolution and is controlled by a family of cysteine proteases. These enzymes cleave after an aspartate residue in their specific substrate, thus mediating many of the typical biochemical and morphological changes that characterize apoptotic cells. In healthy cells, the caspases exist in mitochondria and cytosol as their inactive proenzymes (Mancini, M., et al., J. Cell Biol. 140: 1485-1495 (1998)). Apoptotic signals are transduced along two major pathways: an intrinsic pathway associated with the mitochondria and an extrinsic pathway mediated by death receptors of the tumor necrosis factor receptor superfamily. This cascade can be triggered by a number of different types of stimuli. Agents that damage DNA, such as irradiation and chemotherapeutic agents, activate p53, which can stimulate both pathways of apoptosis. Importantly, caspase 3 activation is required for the execution of both pathways. Thus, caspase 3-induced proteolysis has been shown to be a critical event in virtually all cellular apoptotic pathways. All of the current data suggests that defects in apoptosis are a prerequisite of cancer. Cell growth signals induced by unregulated activity of oncoproteins, such as HER-2, or inactivation of tumor suppressor proteins, such as p53, should trigger caspase activation and increase apoptosis. However, human tumors contain mutations in pro-apoptotic genes (leading to their inactivation) (e.g. p53) and/or have increased expression/activity of anti-apoptotic proteins, resulting in a reduction of or inability of a tumor cell's ability to respond to therapeutic modalities.

Cellular targets can be sensitized to apoptosis induction by the antibodies and proteins by simultaneously treating cells with sublethal doses of chemotherapeutic agents, radiation, or severe hypoxia. These treatments, which can produce an excellent anticancer effect, can markedly promote the apoptotic effect of the antibodies and proteins when used in combination, to produce a synergistic effect. In addition, chemotherapeutic agents may be used in doses much smaller than the usual dose in combination with the antibodies and proteins to produce a satisfactory anticancer effect. This is beneficial because the adverse effects of the chemotherapeutic agent are minimized.

Chemotherapeutic agents that may be used include agents that directly cross-link DNA, agents that intercalate into DNA, and agents that lead to chromosomal and mitotic aberrations by affecting nucleic acid synthesis. Such agents include:

(i) Antibiotics, such as Doxorubicin hydrochloride (5,12-Naphthacenedione, (8s-cis)-10-[(3-amino-2,3,6-trideoxy-a-L-lyxo-hexopyranosyl)oxy]-7,8,9,10-tetrahydro-6,8,11-trihydroxy-8-(hydroxyacetyl)-1-methoxy-hydrochloride) (hydroxydaunorubicin hydrochloride, Adriamycin); Daunorubicin hydrochloride (5,12-Naphthacenedione, (8S-cis)-8-acetyl-10-[(3-amino-2,3,6-trideoxy-a-L-lyxo-hexanopyranosyl)oxy]-7,8,9,10-tetrahydro-6,8,11-trihydroxy-10-methoxy-, hydrochloride); Mitomycin (also known as mutamycin and/or mitomycin-C); Actinomycin D (Dactinomycin); Bleomycin;

(ii) Plant Alkaloids, such as Taxol; Vincristine; Vinblastine;

(iii) Alkylating Agents, such as Carmustine (3 bis(2-chloroethyl)-1-nitrosourea); Melphalan (4-[bis(2-chloroethyl)amino]-L-phenylalanine); Cyclophosphamide(2H-1,3,2-Oxazaphosphorin-2-amine, N,N-bis(2-chloroethyl)tetrahydro-, 2-oxide, monohydrate); Chlorambucil (4-[bis(2-chlorethyl)amino]benzenebutanoic acid); Busulfan (1,4-butanediol dimethanesulfonate); Lomustine (1-(2-chloro-ethyl)-3-cyclohexyl-1 nitrosourea); and

(iv) Miscellaneous Agents, including Cisplatin; VP16 (etoposide); and Tumor Necrosis Factor.

In general, methods of administering antibodies and proteins are well known in the art. In particular, the routes of administration already in use for antibodies and proteins therapeutics, along with formulations in current use, provide preferred routes of administration and formulation for the antibodies and proteins identified as described above.

Antibody and protein compositions can be administered by a number of routes including, but not limited to: oral, intravenous, intraperitoneal, intramuscular, transdermal, subcutaneous, topical or sublingual means. Antibodies and proteins can also be administered via liposomes. Such administration routes and appropriate formulations are generally known to those of skill in the art.

Administration of the formulations described herein may be accomplished by any acceptable method which allows the antibody or protein to reach its target. The particular mode selected will depend of course, upon factors such as the particular formulation, the severity of the state of the subject being treated, and the dosage required for therapeutic efficacy. As generally used herein, an “effective amount” of an antibody or protein is that amount which is able to treat one or more symptoms of disease, reverse the progression of one or more symptoms of disease, halt the progression of one or more symptoms of disease, or prevent the occurrence of one or more symptoms of disease in a subject to whom the formulation is administered, as compared to a matched subject not receiving the compound or therapeutic agent. In a preferred embodiment, the disease is cancer. The actual effective amounts of drug can vary according to the specific drug or combination thereof being utilized, the particular composition formulated, the mode of administration, and the age, weight, condition of the patient, and severity of the symptoms or condition being treated. In determining the effective amount of the antibody or protein to be administered in the treatment or prophylaxis of disease the physician evaluates circulating plasma levels, formulation toxicities, and progression of the disease.

Any acceptable method known to one of ordinary skill in the art may be used to administer a formulation to the subject. The administration may be localized (i.e., to a particular region, physiological system, tissue, organ, or cell type) or systemic, depending on the condition being treated.

Injections can be e.g., intravenous, intradermal, subcutaneous, intramuscular, or intraperitoneal. The composition can be injected intradermally for treatment or prevention of cancer, for example. In some embodiments, the injections can be given at multiple locations. Implantation includes inserting implantable drug delivery systems, e.g., microspheres, hydrogels, polymeric reservoirs, cholesterol matrixes, polymeric systems, e.g., matrix erosion and/or diffusion systems and non-polymeric systems, e.g., compressed, fused, or partially-fused pellets. Inhalation includes administering the composition with an aerosol in an inhaler, either alone or attached to a carrier that can be absorbed. For systemic administration, it may be preferred that the composition is encapsulated in liposomes.

Other delivery systems suitable include, but are not limited to, time-release, delayed release, sustained release, or controlled release delivery systems. Such systems may avoid repeated administrations in many cases, increasing convenience to the subject and the physician. Many types of release delivery systems are available and known to those of ordinary skill in the art. The formulation may be as, for example, microspheres, hydrogels, polymeric reservoirs, cholesterol matrices, or polymeric systems. In some embodiments, the system may allow sustained or controlled release of the composition to occur, for example, through control of the diffusion or erosion/degradation rate of the formulation containing the antibody or protein.

Dosages for a particular patient can be determined by one of ordinary skill in the art using conventional considerations, (e.g. by means of an appropriate, conventional pharmacological protocol). A physician may, for example, prescribe a relatively low dose at first, subsequently increasing the dose until an appropriate response is obtained. The dose administered to a patient is sufficient to effect a beneficial therapeutic response in the patient over time, or, e.g., to reduce symptoms, or other appropriate activity, depending on the application. The dose is determined by the efficacy of the particular formulation, and the activity, stability or serum half-life of the antibody or protein employed and the condition of the patient, as well as the body weight or surface area of the patient to be treated. The size of the dose is also determined by the existence, nature, and extent of any adverse side-effects that accompany the administration of a particular vector, formulation, or the like in a particular patient.

Therapeutic compositions comprising one or more antibodies or proteins are optionally tested in one or more appropriate in vitro and/or in vivo animal models of disease, to confirm efficacy, tissue metabolism, and to estimate dosages, according to methods well known in the art. In particular, dosages can be initially determined by activity, stability or other suitable measures of treatment vs. non-treatment (e.g., comparison of treated vs. untreated cells or animal models), in a relevant assay. Formulations are administered at a rate determined by the LD50 of the relevant formulation, and/or observation of any side-effects of the nucleic acids at various concentrations, e.g., as applied to the mass and overall health of the patient. Administration can be accomplished via single or divided doses.

Examples of such approaches include the use of antibodies such as RITUXAN® directed against the CD20 antigen on the surface of B cells. This approach has been approved by the FDA and represents a major form of therapy against lymphoma.

1. A method for identifying antibodies that induce cell death comprising
(1) exposing an antibody or protein library to multiple isolated normal and abnormal cell populations and
(2) screening for antibodies and proteins that kill the abnormal but not the normal cell populations.

2. The method of claim 1 wherein the cells are in microarrays.

3. The method of claim 1 wherein the antibody library is selected from the group consisting of an animal antibody library, a human antibody library, a synthetic antibody library, a single chain antibody library, and antibodies obtained from the serum of one or more individuals.

4. The method of claim 1 wherein the abnormal cells are cancer cells.

5. The method of claim 1 wherein the antibodies or proteins are identified when a sensitizer such as a cytotoxic compound or immunomodulatory compound is added to the microarray during screening.

6. A method of claim 1 wherein cell death is caused by apoptosis, the complement cascade, or Natural Killer (NK) cells.

7. Antibodies and proteins isolated by the method of claim 1.

8. A method of treating an individual with cancer comprising administering the antibodies and proteins of claim 5 in a pharmaceutically acceptable carrier.
